(19) United States
(12) Patent Application Publication
(10) Pub. No.: US 2009/0304603 A1
(43) Pub. Date: Dec. 10, 2009

(54) TOPICAL STEROID SPRAY WITH BOTANIC SEED OILS

(75) Inventor: Charles E. Crutchfield, III, Mendota Heights, MN (US)

Correspondence Address: BRIGGS AND MORGAN P.A., 2200 IDS CENTER, 80 SOUTH 8TH ST, MINNEAPOLIS, MN, 55402, US

(73) Assignee: CUTICEUTICALS, INC., St. Paul, MN (US)

(21) Appl. No.: 12/539,807

(22) Filed: Aug. 12, 2009

Related U.S. Application Data
(63) Continuation-in-part of application No. 11/930,622, filed on Oct. 31, 2007.
Application No. 11/930,622 is a continuation-in-part of application No. 10/462,458, filed on Jun. 16, 2003, now abandoned.
Application No. 10/462,458 is a continuation-in-part of application No. 09/882,965, filed on Jun. 15, 2001, now Pat. No. 6,579,512.

(51) Int. Cl. A61K 36/00; A61P 29/00; A61K 36/45; A61K 36/73; A61K 36/87; A61K 36/42; A61P 17/00

(52) U.S. Cl. 424/43; 424/725; 424/732; 424/765; 424/766; 424/758

(57) ABSTRACT

A pharmaceutical topical spray composition including a topical corticosteroid, an alcohol, a propellant, isopropyl myristate, and a blend of botanic seed oils is described. The blend of botanic seed oils may include three or more botanic seed oils prepared using a cold press method. A method for treating an inflammatory skin condition by administering the pharmaceutical topical spray composition to the skin of a mammal is also described. The pharmaceutical composition is effective in the treatment of inflammatory skin conditions without the need for zinc pyrithione, undecylenic acid, or a detergent.

### CROSS REFERENCE TO RELATED APPLICATIONS

This application is a continuation-in-part of U.S. patent application Ser. No. 11/930,622, filed Oct. 31, 2007, which is a continuation-in-part of U.S. patent application Ser. No. 10/462,458, filed Jun. 16, 2003, now abandoned, which is a continuation-in-part of U.S. patent application Ser. No. 09/882,965, filed Jun. 15, 2001, now U.S. Pat. No. 6,579,512, the contents of which applications are hereby incorporated by reference in their entireties.

### BACKGROUND OF THE INVENTION

The present invention relates to a pharmaceutical composition for the treatment of inflammatory skin conditions, and a therapeutic method for treating inflammatory skin conditions using the pharmaceutical composition.

Topical corticosteroids are powerful tools for treating skin disease. Understanding the correct use of these agents will result in the successful management of a variety of skin problems. There are many products available, and new ones appear almost monthly. Pharmaceutical companies have responded to the great demand for these agents with an increasing number of products, but all of these preparations have basically the same anti-inflammatory properties. They differ only in strength, base and price.

The anti-inflammatory properties of topical corticosteroids result in part from their ability to induce vasoconstriction of the small blood vessels in the upper dermis. This property is used in an assay procedure to determine the strength of each new product. These products are subsequently tabulated in seven groups, with group I the strongest and group VII the weakest (see the Formulary in Table 1, below).

TABLE 1
Steroid Strength by Group Number

| Group | |
|---|---|
| No. | Generic Name |
| I | Clobetasol propionate |
| II | Fluocinonide |
| III | Triamcinolone acetonide |
| IV | Fluocinolone acetonide |
| V | Hydrocortisone valerate |
| VI | Desonide |
| VII | Hydrocortisone |

This treatment recommends topical steroids by group number rather than by generic or brand name because the agents in each group are essentially equivalent in strength. When a new topical corticosteroid appears on the market, ask to which group it belongs and add it to the list in the Formulary.

Guidelines for choosing the appropriate strength of topical steroid are presented in the chart below (Table 2).

TABLE 2
Suggested Strengths of Topical Steroids for Various Conditions
SUGGESTED STRENGTH OF TOPICAL STEROIDS TO
INITIATE TREATMENT*

| GROUPS I-II | GROUPS III-V | GROUPS VI-VII |
|---|---|---|
| Psoriasis | Atopic dermatitis | Dermatitis (eyelids) |
| Lichen planus | Nummular eczema | Dermatitis (diaper area) |
| Discoid lupus† | Asteatotic eczema | Mild dermatitis (face) |
| Severe hand eczema | Stasis dermatitis | Mild anal |
| | | inflammation |
| Poison ivy (severe) | Seborrheid dermatitis | Mild intertrigo |
| Lichen simplex chronicus | Lichen sclerosis et atrophicus | |
| | (vulva) | |
| Hyperkeratotic eczema | Intertrigo (brief course) | |
| Chapped feet | Tinea (brief course to control | |
| | inflammation) | |
| Lichen sclerosis et atrophicus | Scabies (after scabicide) | |
| (skin) | | |
| Alopecia areata | Intertrigo (severe cases) | |
| Nummular eczema (severe) | Anal inflammation (severe | |
| | cases) | |
| Atopic dermatitis (resistant | Severe dermatitis (face) | |
| adult cases) | | |

*Stop treatment, change to less potent agent, or use intermittent treatment once inflammation is controlled.
†Use on the face may be justified.

The best results are obtained when preparations of adequate strength are used for a specified length of time. Weaker, “safer” strengths often fail to provide adequate control. Patients who do not respond after 1 to 4 weeks of treatment should be reevaluated.

Additionally, topical preparations of the steroid clobetasol propionate are indicated for the relief of the inflammatory and pruritic manifestations of cortico-steroid-responsive dermatosis. See, for example, Maloney, et al., “Clobetasol Propionate Emollient 0.05% in the Treatment of Atopic Dermatitis”, International J. of Dermatology, 1998, 37, 128-144.

In the past, it has been found that clobetasol propionate is most effective in the treating of inflammatory skin conditions when combined with zinc pyrithione and undecylenic acid. For example, Seidel (U.S. Pat. No. 5,972,920) discloses the use of clobetasol propionate in combination with either zinc pyrithione, undecylenic acid, or both. Applicant Crutchfield also noted the requirement for zinc pyrithione in Crutchfield, et. al., “The Effective Use of Topical Zinc Pyrithione in the Treatment of Psoriasis: a Report of Three Cases”, J. Geriatr. Dermatol. 1997; 5(1):21-4

Surprisingly, the applicant has found that zinc pyrithione and undecylenic acid are not necessary for the optimal effectiveness of clobetasol propionate.

Studies have also indicated that some sort of surfactant, such as sodium lauryl sulfate, is necessary for the optimal effectiveness of clobetasol propionate, whether alone or combined with zinc pyrithione and undecylenic acid. Again, Seidel '920 discloses the use of an anionic surfactant (sodium lauryl sulfate) in conjunction with clobetasol propionate, zinc pyrithione, and undecylenic acid.

Surprisingly, the applicant has found that no surfactant is necessary for the optimal effectiveness of clobetasol propionate.

Applicants have also found that the composition is most effective and easily tolerated by patients when administered in a spray form by means of a propellant. In contrast, Seidel '920 teaches away from the use of a spray as being highly evaporative and producing a painful freezing sensation to the skin and that some propellants are explosive.

### SUMMARY OF THE INVENTION

The present invention is directed to a pharmaceutical topical spray composition including a topical corticosteroid, an alcohol, a propellant, and isopropyl palmitate. The present invention is also directed to a pharmaceutical topical spray composition including botanic seed oils. Specifically, an embodiment of the pharmaceutical topical spray composition includes a topical corticosteroid, an alcohol, a propellant, and botanic seed oils including a blend of black raspberry seed oil, black cumin seed oil, and red raspberry seed oil. Another embodiment of the pharmaceutical topical spray composition includes a topical corticosteroid, an alcohol, a propellant, isopropyl myristate, and a blend of botanic seed oils. This blend of botanic seed oils may, for example, include three or more botanic seed oils that are prepared according to a cold press method.

The present invention is also directed to a method for treating an inflammatory skin condition by administering a pharmaceutical topical spray composition including a topical corticosteroid to the skin of a mammal. The pharmaceutical composition is effective in the treatment of inflammatory skin conditions without the need for zinc pyrithione, undecylenic acid, or a detergent.

An object and advantage of the present invention is the use of a composition including both a corticosteroid and a blend of botanic seed oils in the treatment of skin conditions.

Another object and advantage of the present invention is the use of a composition including blends of two or more botanic seed oils in the treatment of skin conditions.

Another object and advantage of the present invention is the use of a composition that is effective in the treatment of inflammatory skin conditions and does not include zinc pyrithione, undecylenic acid, or a detergent.

Other advantages will be understood from reading the Detailed Description of the Preferred Embodiments.

The foregoing has outlined rather broadly the features and technical advantages of the present invention in order that the detailed description of the invention that follows may be better understood. Additional features of the invention which form the subject of the claims of the invention will be described hereinafter. It should be appreciated by those skilled in the art that the specific embodiments disclosed may be readily utilized as a basis for modifying or designing other methods or compositions for carrying out the same purposes of the present invention. It should also be realized by those skilled in the art that such equivalent compositions do not depart from the spirit and scope of the invention as set forth in the appended claims. The novel features which are believed to be characteristic of the invention, both as to its composition and method of operation, together with further objects and advantages will be better understood from the following description.

### DETAILED DESCRIPTION OF THE PREFERRED EMBODIMENTS

A first pharmaceutical composition for the treatment of inflammatory skin conditions includes clobetasol propionate, an alcohol, a propellant, and isopropyl palmitate, suitable for topical administration. A second pharmaceutical composition for the treatment of inflammatory skin conditions includes clobetasol propionate, an alcohol, a propellant, and a blend of black raspberry and black cumin seed oils, sold under the trademark Immuno-Viva®, a trademark owned by Botanic Oil Innovations, Inc., and further blended with red raspberry seed oil, and suitable for topical administration. A third pharmaceutical composition for the treatment of inflammatory skin conditions includes a topical corticosteroid, an alcohol, a propellant, isopropyl myristate, and a blend of botanic seed oils, suitable for topical administration. These pharmaceutical compositions are suitably carried in an aerosol can with a nozzle. Applicants have found that no other active ingredients are necessary for the optimal pharmaceutical action of the topical corticosteroids in these compositions.

Starting with the first pharmaceutical composition, preferably, the clobetasol propionate is present in about 0.01% to 10% (% w/w). More preferably, the clobetasol propionate is present in about 0.01% to 1% (% w/w). Most preferably, the clobetasol propionate is present in the amount of 0.05% (% w/w). Any of the above corticosteroids may be used in this spray formulary.

Any number of alcohols can be used as a solvent in the preparation, such as methanol, ethanol, propanol, isopropanol, butanol, or isobutanol. However, Applicants have found that denatured ethanol (SDA-40 200 proof) is a suitable and effective alcohol to use in the composition. Most preferably, the ethanol is present in the amount of 37.43% (% w/w). However, a range of 27% (% w/w) to 47% (% w/w) may be used while a narrower range of 32% (% w/w) to 42% (% w/w) is more suitable.

The composition also contains isopropyl palmitate as an emollient oil or carrier most preferably in the amount of 37.72% (% w/w). However, a range of 27% (% w/w) to 47% (% w/w) may be used while a narrower range of 32% (% w/w) to 42% (% w/w) is more suitable. The alcohol and oil is about a 50:50 mixed blend.

An inactive ingredient, but one of importance in delivery of the composition to the skin, is a propellant. Any propellant conventionally used in the delivery of aerosol sprays may be used. Most preferably, an amount of 24.51% (% w/w) of isobutane is in the composition. However, a range of 20% (% w/w) to 30% (% w/w) may also be suitable.

A small amount of water, approximately 0.22%, is appropriate.

Importantly, the composition does not contain either zinc pyrithione or undecylenic acid.

A therapeutic method for treating an inflammatory skin condition comprises administering the above first composition to the skin of a mammal in need of such therapy.

The second pharmaceutical composition differs significantly from the first pharmaceutical composition by using concentrated plant seed oils, also referred to as botanic seed oils prepared according to a cold press method, as the emollient oil or vehicle. The third pharmaceutical composition uses both isopropyl myristate and botanic seed oils as emollient oils or vehicles. Plant seed oils are an excellent source of antioxidants. In addition to traditional antioxidants, such as vitamins C and E, plant seed oils contain phenolic compounds which are excellent free radical scavengers. Black raspberry and red raspberry seed oils have a diversity and ultra-rich content of antioxidants, including 4 different forms of Vitamin E (Alpha and Gamma Tocopherol, Beta and Gamma Tocotrienol). These raspberry seed oils contain Omega 3 and Omega 6, In U.S. Patent Publication No. 20070243310, Synergistic super potent antioxidant cold pressed botanic oil blends, to Leonard et al., published Oct. 18, 2007, the inventors describe blends of seed oils as having a synergistic antioxidant effect.

Concentrated seed oils are described as immunostimulants in U.S. Patent Publication No. 2007/0128301, Immune Enhancement by Seed Oil and/Or Seed Flour, to Saltzman et al., published Jun. 7, 2007, and for use in treatment of cancer in Patent Publication No. 2005/0244375, Composition and Method of Cancer Treatment, to Leonard et al., published Nov. 3, 2005. In Saltzman et al., the applicant theorized that concentrated seed oils possess anti-inflammatory properties.

The use of concentrated seed oils in the pharmaceutical composition acts as natural emollients to prevent dryness and protect the skin, acting as a barrier and healing agent, as well as a soothing and softening agent of the skin. The concentrated seed oils in the pharmaceutical composition can reduce roughness, cracking and irritation. The use of seed oil emollients nourish the skin with concentrated nutrients that can be beneficial in treating inflammatory conditions of the skin such as acne, psoriasis, eczema and rosacea. The antioxidant properties of botanic seed oils may prove to have some of the most effective rejuvenating properties of any known skin treatment to date. Charles E. Crutchfield, M.D. Assoc. Prof. of Dermatology, University of Minnesota, et al., The Use of Nature Fresh Cold Pressed Seed Oils for Skin and Personal Care Products—A New Approach (an article pending publication).

In The Use of Nature Fresh Cold Pressed Seed Oils for Skin and Personal Care Products—A New Approach article, there is further discussion about research showing that synergistic botanic seed oils can inhibit Cox-2 activity. Cox-2 is an enzyme linked to inflammation and inflammation is associated with many skin disorders ranging from sunburn to rosacea, psoriasis, acne and dandruff. In addition, the article discusses research that synergistic botanic seed oils have potent antimicrobial activity. Many skin disorders are known to result from or be exacerbated by bacteria and fungi living on the skin surface.

A preferred method of preparing the seed oils, used in the invention, is described in U.S. Patent Publication No. 2007/0128301, Immune Enhancement by Seed Oil and/Or Seed Flour, Saltzman et al., published Jun. 7, 2007, U.S. Patent publication is incorporated by reference herein in its entirety. That description is:
The oils for the composition are prepared from seeds which have been carefully dried and cleaned at temperatures below 120 degrees F. In a cold press process, the seeds are fed through the press and put under high pressure with no extra heat during the pressing process. Oil temperatures during extraction are typically 70 degrees to 90 degrees F. To insure minimal or no oxidation and the highest potential antioxidant level of the oils, the press head and oil extraction chamber can be enclosed within an inert atmosphere.

Refining or removal of suspended solids and container filling can also be done in an inert atmosphere to preserve quality.

U.S. Patent Publication No. 2007/0128301, paragraph [0016].

The second pharmaceutical composition for the treatment of inflammatory skin conditions includes clobetasol propionate, an alcohol, a propellant, and botanic seed oils including a blend of black raspberry oil, black cumin seed oil, and red raspberry seed oil, suitable for topical administration.

Preferably, in the second pharmaceutical composition the clobetasol propionate is present in about 0.01 to 10% (% w/w). More preferably, the clobetasol propionate is present in about 0.01% to 1% (% w/w). Most preferably, the clobetasol propionate is present in the amount of 0.05% (% w/w). Any of the above corticosteroids may be used in this spray formulary.

As discussed above, clobetasol propionate, a Group I topical corticosteroid, is a preferred steroid. However, weaker topical corticosteroids in Groups II through VII can be used in the spray formulary with an adjustment in the weight for a therapeutically effective replacement for clobetasol propionate. This involves increasing the topical steroid amount to compensate for weaker activity. Whereas, the clobetasol propionate is more preferably present in about 0.01% to 1% (% w/w), the weaker topical corticosteroids in Groups II through VII can be used more preferably in an amount of about 0.01% to 2.5% (% w/w). The increase in the amount of the topical corticosteroid would result in a decrease in the amount of emollient seed oils. A person of skill in the art would be able to determine the appropriate effective therapeutic amount for replacement with another topical steroid of different strength.

Any number of alcohols can be used as a solvent in the preparation, such as methanol, ethanol, propanol, isopropanol, butanol, or isobutanol. However, Applicants have found that denatured ethanol (SDA-40 200 proof) is a suitable and effective alcohol to use in the composition. Most preferably, the ethanol is present in the amount of 37.43% (% w/w). However, a range of 27% (% w/w) to 47% (% w/w) may be used while a narrower range of 32% (% w/w) to 42% (% w/w) is more suitable.

The second pharmaceutical composition contains botanic seed oils including a blend of black raspberry seed oil, black cumin seed oils, and red raspberry seed oil as an emollient oil or carrier. The blend of black raspberry oil and black cumin seed oil sold as Immuno-Viva® has a ratio of about 50:50.

An embodiment of the second pharmaceutical composition used to treat patients with psoriasis employed a blend of botanic seed oils is in the amount of about 8.96% (% w/w) black raspberry seed oil, about 8.96% (% w/w) black cumin seed oil, about 17.92% (% w/w) red raspberry seed oil, 0.27% (% w/w) pumpkin seed oil, 0.27% (% w/w) chardonnay grape seed oil, 0.27% (% w/w) carrot seed oil, 0.27% (% w/w) blueberry seed oil, 0.27% (% w/w) cranberry seed oil, 0.27% (% w/w) pomegranate seed oil that makes up a total seed oil blend of about 37.72% (% w/w). The ratio of the blend of black raspberry and black cumin seed oils, sold under Immuno-Viva®, to the red raspberry oil was about 47.5:47.5. It is preferable that the ratio of black raspberry and black cumin seed oils to red raspberry seed oil be about 47.5 to 47.5, and this portion of the seed oil blend be about 36% (% w/w). The other amounts of seed oil, about 2% (% w/w), make up the remainder of the complete blend to be about 38% (% w/w).

A range of 27% (% w/w) to 47% (% w/w) of the complete blend of seed oils in the above proportions can be used, while a narrower range of 32% (% w/w) to 42% (% w/w) in the above proportions is more suitable.

Blends of two or more of the concentrated seed oils are preferred to use of a single seed oil. A preferred composition involves a blend including three concentrated seed oils in the pharmaceutical composition as the predominant component of this seed oil blend having synergistic antioxidant properties.

Not to be bound by theory, but blends of concentrated seed oils in the present invention are theorized to work with topical steroids such as clobetasol propionate, to increase the healing effectiveness of the topical steroid when used on inflammatory skin conditions.

An inactive ingredient, but one of importance in delivery of the composition to the skin, is a propellant. Any propellant conventionally used in the delivery of aerosol sprays may be used. Most preferably, an amount of 24.51% (% w/w) of isobutane is in the composition. However, a range of 20% (% w/w) to 30% (% w/w) may also be suitable.

A small amount of water, approximately 0.22%, is appropriate.

The composition does not contain either zinc pyrithione or undecylenic acid.

Under the inventor's supervision, 1,450 patients with an inflammatory skin condition, psoriasis, were treated with a preferred embodiment of the second pharmaceutical composition having clobetasol propionate, an alcohol, a propellant, and botanic seed oils in a blend of black raspberry seed oil, black cumin seed oils, red raspberry seed oil, pumpkin seed oil, chardonnay grape seed oil, carrot seed oil, blueberry seed oil, cranberry seed oil, and pomegranate seed oil. The results showed that 96% of the patents reported significant improvement using the second pharmaceutical composition.

A therapeutic method for treating an inflammatory skin condition comprises administering the second pharmaceutical composition to the skin of a mammal in need of such therapy.

The third pharmaceutical composition for the treatment of inflammatory skin conditions consists of a topical corticosteroid, an alcohol, a propellant, isopropyl myristate, and a blend of botanic seed oils, suitable for topical administration.

Clobetasol propionate, a Group I topical corticosteroid, is a preferred topical corticosteroid for use in the third pharmaceutical composition. However, weaker topical corticosteroids in Groups II through VII, such as the steroids listed in Table 1, may also be used. For example, hydrocortisone is a preferred weaker corticosteroid for use in the third pharmaceutical composition. While clobetasol propionate may be used in a prescription strength version of the third composition, hydrocortisone may be used in an over-the-counter version of the third composition.

Preferably, if clobetasol propionate is used in the third pharmaceutical composition, the clobetasol propionate is present in an amount of about 0.01% to about 10% (% w/w). More preferably, the clobetasol propionate is present in an amount of about 0.01% to about 1% (% w/w). Most preferably, the clobetasol propionate is present in an amount of about 0.04% (% w/w).

When weaker corticosteroids than clobetasol propionate are used in the third composition, the weight percentage of the corticosteroid in the composition may be adjusted to provide a therapeutically effective replacement for clobetasol propionate. This involves increasing the topical steroid amount to compensate for weaker activity. Whereas the clobetasol propionate is more preferably present in an amount of about 0.01% to about 1% (% w/w), the weaker topical corticosteroids in Groups II through VII can be used more preferably in an amount of about 0.01% to about 2.5% (% w/w). The increase in the amount of the topical corticosteroid would result in a decrease in the amount of one or more of the other ingredients in the composition. A person of skill in the art would be able to determine the appropriate effective therapeutic amount for a topical steroid of different strength. If hydrocortisone is used in the third pharmaceutical composition, the hydrocortisone is preferably present in an amount of about 0.01% to about 10% (% w/w). More preferably, the hydrocortisone is present in an amount of about 0.01% to about 2.5% (% w/w). Most preferably, the hydrocortisone is present in an amount of about 0.75% (% w/w).

Any number of alcohols can be used as a solvent in the third composition, such as methanol, ethanol, propanol, isopropanol, butanol, or isobutanol. However, Applicants have found that denatured ethanol (SDA-40 200 proof) is a suitable and effective alcohol to use in the composition. Preferably, the ethanol is present in an amount of about 20% to about 40% (% w/w). More preferably, the ethanol is present in an amount of about 25% to about 35% (% w/w). Most preferably, the ethanol is present in an amount of about 30% (% w/w).

The isopropyl myristate is preferably present in the third composition in an amount ranging from about 13% to about 33% (% w/w), and is more preferably present in an amount ranging from about 18% to about 28% (% w/w). Most preferably, the isopropyl myristate is present in an amount of about 22% to about 23% (% w/w).

The blend of botanic seed oils used in the third composition may include one or more of a variety of botanic seed oils, such as red raspberry seed oil, black raspberry seed oil, blueberry seed oil, blackberry seed oil, carrot seed oil, grape seed oil, cranberry seed oil, pomegranate seed oil, pumpkin seed oil, and black cumin seed oil. Not to be bound by theory, but blends of concentrated seed oils in the present invention are theorized to work with topical steroids such as clobetasol propionate, to increase the healing effectiveness of the topical steroid when used on inflammatory skin conditions. Blends of two or more of the concentrated seed oils are preferred to use of a single seed oil. A preferred composition involves a blend including three concentrated seed oils in the pharmaceutical composition as the predominant component of this seed oil blend having synergistic antioxidant properties.

In an embodiment of the third pharmaceutical composition, the majority of the botanic seed oil blend is composed of red raspberry seed oil, while the remainder of the blend is composed of equal amounts of one or more botanic seed oils selected from the following seed oils: black raspberry seed oil, blueberry seed oil, blackberry seed oil, carrot seed oil, grape seed oil, cranberry seed oil, pomegranate seed oil, pumpkin seed oil, and black cumin seed oil. In a preferred embodiment, all of the botanic seed oils listed above are used in the botanic seed oil blend. For example, in a preferred embodiment of the botanic seed oil blend, the red raspberry seed oil is present in an amount of about 82% to about 98.2% (% w/w), while black raspberry seed oil blueberry seed oil, blackberry seed oil, carrot seed oil, grape seed oil, cranberry seed oil, pomegranate seed oil, pumpkin seed oil, and black cumin seed oil are each present in an amount ranging from about 0.2% to about 2% (% w/w). More preferably, the red raspberry seed oil is present in an amount ranging from about 86.5% to about 95.5% (% w/w), while the remaining seed oils are each present in an amount ranging from about 0.5% to about 1.5% (% w/w). A formula of a botanic seed oil blend that may be used in the third pharmaceutical composition is provided in Table 3.

TABLE 3
Botanic Seed Oil Blend (per 100 grams)

| Botanic Seed Oil | Amount (in grams) |
|---|---|
| Red Raspberry Seed Oil | 91 |
| Blueberry Seed Oil | 1 |
| Blackberry Seed Oil | 1 |
| Carrot Seed Oil | 1 |
| Chardonnay Grape Seed Oil | 1 |
| Black Raspberry Seed Oil | 1 |
| Cranberry Seed Oil | 1 |
| Pomegranate Seed Oil | 1 |
| Pumpkin Seed Oil | 1 |
| Black Cumin Seed Oil | 1 |

Preferably, the blend of botanic seed oils is present in the third composition in an amount ranging from about 13% to about 33% (% w/w). More preferably, the blend of botanic seed oils is present in an amount ranging from about 18% to about 28% (% w/w). Most preferably, the blend of botanic seed oils is present in an amount of about 22% to about 23% (% w/w).

An inactive ingredient, but one of importance in delivery of the composition to the skin, is a propellant. Any propellant conventionally used in the delivery of aerosol sprays may be used. Preferably, a blend of butane and propane is used as the propellant. For example, a blend of propane and normal butane, a blend of propane and isobutane, or a blend of propane, isobutane and normal butane, mixed to have a vapor pressure of approximately 46 psig at 70° F., may be used. Most preferably, the blend of butane and propane is present in the composition in an amount of about 24.51% (% w/w). However, a range of 20% (% w/w) to 30% (% w/w) may also be suitable.

The third composition does not contain either zinc pyrithione or undecylenic acid.

A formula of a prescription strength embodiment of the third pharmaceutical composition, in which the combined amount of ethanol, isopropyl myristate, botanic seed oils, and clobetasol propionate totals 100 grams, is provided in Table 4.

TABLE 4
Example of Prescription Strength Aerosol Spray

| | Amount (in | Percentage |
|---|---|---|
| Ingredient | grams) | (% w/w) |
| 200 proof ethanol | 39.975 | 30.177 |
| Isopropyl myristate | 29.9875 | 22.638 |
| Botanic seed oil blend (formulation | 29.9875 | 22.638 |
| provided in Table 3) | | |
| Clobetasol propionate micronized | 0.050 | 0.038 |
| powder | | |
| B46 propellant (blend of propane and | 32.468 | 24.510 |
| normal butane mixed to have a vapor | | |
| pressure of 46 psig at 70° F.) | | |

A formula of an over-the-counter strength embodiment of the third pharmaceutical composition, in which the combined amount of ethanol, isopropyl myristate, botanic seed oils, and hydrocortisone totals 100 grams, is provided in Table 5.

TABLE 5
Example of Over-the-Counter Strength Aerosol Spray

| | Amount (in | Percentage |
|---|---|---|
| Ingredient | grams) | (% w/w) |
| 200 proof ethanol | 40.00 | 30.20 |
| Isopropyl myristate | 29.50 | 22.27 |
| Botanic seed oil blend (formulation | 29.50 | 22.27 |
| provided in Table 3) | | |
| Hydrocortisone micronized powder | 1.00 | 0.755 |
| B46 propellant (blend of propane and | 32.47 | 24.51 |
| normal butane mixed to have a vapor | | |
| pressure of 46 psig at 70° F.) | | |

A therapeutic method for treating an inflammatory skin condition comprises administering the third pharmaceutical composition to the skin of a mammal in need of such therapy.

The present invention may be embodied in other specific forms without departing from the spirit or essential attributes thereof, and it is therefore desired that the present embodiments be considered in all respects as illustrative and not restrictive, reference being made to the appended claims rather than to the foregoing description to indicate the scope of the invention.

What is claimed is:

1. A therapeutic method for treating an inflammatory skin condition, comprising:
administering by spray to the skin of a mammal in need of such therapy, an effective amount of a pharmaceutical composition comprising a topical corticosteroid, an alcohol, isopropyl myristate, and a blend of three or more botanic seed oils that are prepared according to a cold press method.

2. The therapeutic method of claim 1, wherein the topical corticosteroid is selected from the group consisting of clobetasol propionate, fluocinonide, triamcinolone acetonide, fluocinolone acetonide, hydrocortisone valerate, desonide, and hydrocortisone.

3. The therapeutic method of claim 1, wherein the topical corticosteroid is present in an amount ranging from about 0.01% to about 2.5% (% w/w).

4. The therapeutic method of claim 1, wherein the blend of three or more botanic seed oils comprises three or more botanic seed oils selected from the group consisting of red raspberry seed oil, blueberry seed oil, blackberry seed oil, carrot seed oil, grape seed oil, black raspberry seed oil cranberry seed oil, pomegranate seed oil, pumpkin seed oil, and black cumin seed oil.

5. The therapeutic method of claim 1, wherein the blend of three or more botanic seed oils is present in an amount ranging from about 13% (% w/w) to about 33% (% w/w).

6. The therapeutic method of claim 1, wherein the blend of three or more botanic seed oils is present in an amount ranging from about 18% (% w/w) to about 28% (% w/w).

7. The therapeutic method of claim 1, wherein the inflammatory skin condition is psoriasis, atopic dermatitis, eczema, lupus, poison ivy, scabies, severe skin inflammation, dermatitis, lichen, or papulosquamous.

8. The therapeutic method of claim 1, wherein the administration is performed in the absence of zinc pyrithione and undecylinic acid.

9. The therapeutic method of claim 1, wherein the pharmaceutical composition further comprises a propellant comprising propane and butane.

10. The therapeutic method of claim 9, wherein the propellant is present in an amount ranging from about 20% (% w/w) to about 30% (% w/w).

11. A pharmaceutical topical spray composition, comprising:
a topical corticosteroid;
an alcohol;
isopropyl myristate; and,
a blend of three or more botanic seed oils.

12. The pharmaceutical composition of claim 11, wherein the topical corticosteroid is selected from the group consisting of clobetasol propionate, fluocinonide, triamcinolone acetonide, fluocinolone acetonide, hydrocortisone valerate, desonide, and hydrocortisone.

13. The pharmaceutical composition of claim 11, wherein the topical corticosteroid is present in an amount ranging from about 0.01% to about 2.5% (% w/w).

14. The pharmaceutical composition of claim 11, wherein the blend of three or more botanic seed oils comprises three or more botanic seed oils selected from the group consisting of red raspberry seed oil, blueberry seed oil, blackberry seed oil, carrot seed oil, grape seed oil, black raspberry seed oil, cranberry seed oil, pomegranate seed oil, pumpkin seed oil, and black cumin seed oil.

15. The pharmaceutical composition of claim 11, wherein the blend of three or more botanic seed oils is present in an amount ranging from about 13% (% w/w) to about 33% (% w/w).

16. The pharmaceutical composition of claim 11, wherein the blend of three or more botanic seed oils comprises red raspberry seed oil present in an amount ranging from about 82% to about 98.2% (% w/w) by weight of the blend.

17. The pharmaceutical composition of claim 11, wherein the blend of three or more botanic seed oils comprises botanic seed oils prepared according to a cold press method.

18. The pharmaceutical composition of claim 11, wherein said composition is free of zinc pyrithione and undecylenic acid.

19. The pharmaceutical composition of claim 11, further comprising a propellant comprising propane and butane.

20. The pharmaceutical composition of claim 19, wherein the propellant is present in an amount ranging from about 20% (% w/w) to about 30% (% w/w).

21. A pharmaceutical topical spray composition, comprising:
a topical corticosteroid in an amount ranging from about 0.01% to about 10% (% w/w);
an alcohol in an amount ranging from about 20% to about 40% (% w/w);
isopropyl myristate in an amount ranging from about 13% to about 33% (% w/w);
a blend of three or more botanic seed oils in an amount ranging from about 13% to about 33% (% w/w); and,
a propellant in an amount ranging from about 20% (% w/w) to about 30% (% w/w).

22. The pharmaceutical composition of claim 21, wherein the topical corticosteroid is selected from the group consisting of clobetasol propionate, fluocinonide, triamcinolone acetonide, fluocinolone acetonide, hydrocortisone valerate, desonide, and hydrocortisone.

23. The pharmaceutical composition of claim 21, wherein the topical corticosteroid is present in an amount ranging from about 0.01% to about 2.5% (% w/w).

24. The pharmaceutical composition of claim 21, wherein the blend of three or more botanic seed oils comprises three or more botanic seed oils selected from the group consisting of red raspberry seed oil, blueberry seed oil, blackberry seed oil, carrot seed oil, grape seed oil, black raspberry seed oil, cranberry seed oil, pomegranate seed oil, pumpkin seed oil, and black cumin seed oil.

25. The pharmaceutical composition of claim 21, wherein the blend of three or more botanic seed oils is present in an amount ranging from about 18% (% w/w) to about 28% (% w/w).

26. The pharmaceutical composition of claim 21, wherein the blend of three or more botanic seed oils comprises red raspberry seed oil present in an amount ranging from about 82% to about 98.2% (% w/w) by weight of the blend.

27. The pharmaceutical composition of claim 21, wherein the blend of three or more botanic seed oils comprises botanic seed oils prepared according to a cold press method.

28. The pharmaceutical composition of claim 21, wherein said composition is free of zinc pyrithione and undecylenic acid.
